(19) United States
(12) Patent Application Publication
(10) Pub. No.: US 2009/0240275 A9
(43) Pub. Date: Sep. 24, 2009

(54) Surgical actuator and locking system

(75) Inventor: Ernesto E. Blanco, Belmont, MA (US)

Correspondence Address: OBLON, SPIVAK, MCCLELLAND MAIER & NEUSTADT, P.C., 1940 DUKE STREET, ALEXANDRIA, VA, 22314, US

(73) Assignee: Erblan Surgical Inc., Belmont, MA (US)

(21) Appl. No.: 11/206,864

(22) Filed: Aug. 19, 2005

Related U.S. Application Data
(63) Continuation-in-part of application No. 10/092,560, filed on Mar. 8, 2002.
(60) Provisional application No. 60/608,100, filed on Sep. 9, 2004.

(51) Int. Cl. A61B 17/34

(52) U.S. Cl. 606/185

(57) ABSTRACT

A surgical device including a cutting blade; a guard portion movable with respect to the blade from a first position covering the blade to a second position exposing the blade; an actuator shaft; a biasing element; an integrally formed locking element; and a handle having a cavity configured to receive the locking element.

### CROSS REFERENCE TO RELATED APPLICATIONS

This application claims the benefit of the earlier filing date of U.S. Provisional Application No. 60/608,100, filed Sep. 9, 2004, entitled “Surgical Actuator and Locking System,” as well as to now pending application Ser. No. 10/092,560, filed Mar. 8, 2002, which is a continuation-in-part of application Ser. No. 09/598,453, filed Jun. 22, 2000, now issued Pat. No. 6,497,687, the disclosure of each of which is incorporated by reference herein.

### BACKGROUND OF THE INVENTION

1. Field of the Invention

The device described here is intended specifically for use in surgical systems which require on-off actuators which control function and in turn allow it to occur only once each time a command is given. In addition to that the described system is intended to display the system readiness and the monitoring of its function throughout each cycle. One intended use for this system of locking is its applicability in the control of trocars used in endoscopic surgical procedures such as the one described in U.S. Pat. No. 6,497,687, as well as many other cases where similar results are desired.

2. Description of the Related Art

Most existing trocars used for endoscopic surgical procedures are incapable of truly effective prevention of injuries to internal organs during insertion and manipulation of the trocar. Despite intensive efforts to improve present trocar designs, the results are still disappointing. Present procedures frequently injure internal organs, and the resulting wounds are sometimes serious or even fatal. The need for safer trocars is thus imperative, especially given that endoscopic surgical procedures are likely to become more widespread in the future.

Endoscopic or minimally invasive surgery presents an opportunity to improve present surgical procedures and instrumentation comparable only to the revolutionary effect of the introduction of anesthetics in the 19th Century.

Most present day trocars utilize a tip shield, or cover, for the cutting edges which is usually deployed immediately after penetration of the body cavity has taken place. Such penetration is fraught with danger of injury to internal organs. However careful a surgeon may be during penetration of the body cavity, the resistance to penetration drops at the last instant prior to damage to the internal organs. This sudden drop in the resistance to penetration is called a “plunge effect” and occurs prior to any safety feature deployment. In some trocars, the penetration is controlled in some fashion, either taking place in small increments or under some form of approximate direct observation, estimate, or monitoring. In all cases, however, the designs result in much of the piercing tip being inserted to a dangerous depth before any protecting devices is deployed. This is perhaps not surprising since, after all, a hole must be made before any protection is deployed.

Since in most cases delicate organs are very close to the inside of the skin layer being pierced, it is advisable to perform the penetration after internal cavities have been filled with carbon dioxide to minimize the danger of accidental injury due to contact with the sharp piercing tip or the cutting edges of the instrument. In most cases, however, the force required for penetration and the elastic nature of the muscular layer cause a severe depression at the surgical portal, therefore bringing the penetrating tip of the instrument closer to the internal organs. In some of those cases, the sudden penetration of the cavity wall and the rapid drop in resistance allow the instrument to be propelled far deeper than desired or possible to control. Furthermore, friction between the tissue walls and any protective device retards the deployment of the protective device, and an injury almost inevitably occurs.

Accordingly, a safer surgical device for use in endoscopic procedures is desired.

### SUMMARY OF THE INVENTION

One aspect of the present invention includes a surgical device including a cutting blade; a guard portion movable with respect to the blade from a first position covering the blade to a second position exposing the blade; an actuator shaft extending along an axis of the surgical device from a first end connected to the guard portion to a second end; a biasing element; an integrally formed locking element; and a handle having a cavity configured to receive the locking element.

Another aspect of the present invention includes a method of using a surgical device including depressing an actuating portion to remove a guide portion of a locking element from a locking notch; moving the locking element from a first position to a second position to uncover a penetrator protected by a guard portion connected to the locking element; piercing a membrane such as the peritoneum of a patient with the penetrator; and moving the locking element from the second position to the first position to recover the penetrator with the guard portion.

A further aspect of the present invention includes a method of assembling a surgical device including attaching an integrally formed locking element to an actuator shaft; inserting a biasing element into a hub of the locking element; placing the locking element in a first handle portion with one end of the biasing element facing a surface of the first handle portion; and connecting a second mating handle portion to the first handle portion over the locking element.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a top plan view of a preferred embodiment of the present invention;

FIG. 2 is a side elevational view thereof;

FIG. 3 is a proximal end view thereof;

FIG. 4 is a perspective view of an integrated actuator button and latch module of an embodiment of the present invention;

FIG. 5 is a side cross-sectional view of the present invention;

FIG. 6 is a proximal end cross-sectional view taken along line 6-6 of FIG. 5;

FIG. 7 is a side cross-sectional view of the present invention in a rest position;

FIG. 8 is an end cross-sectional view taken along line 8-8 of FIG. 7;

FIG. 9 is a side cross-sectional view of the present invention in an arming position;

FIG. 10 is an end cross-sectional view taken along line 10-10 of FIG. 9;

FIG. 11 is a side cross-sectional view of the hold downs in the arming position;

FIG. 12 is a side cross-sectional view of the present invention in a release position;

FIG. 13 is an end cross-sectional view taken along line 13-13 of FIG. 12;

### DETAILED DESCRIPTION OF THE INVENTION

Referring now to the drawings, wherein like reference numerals designate identical or corresponding parts throughout the several views.

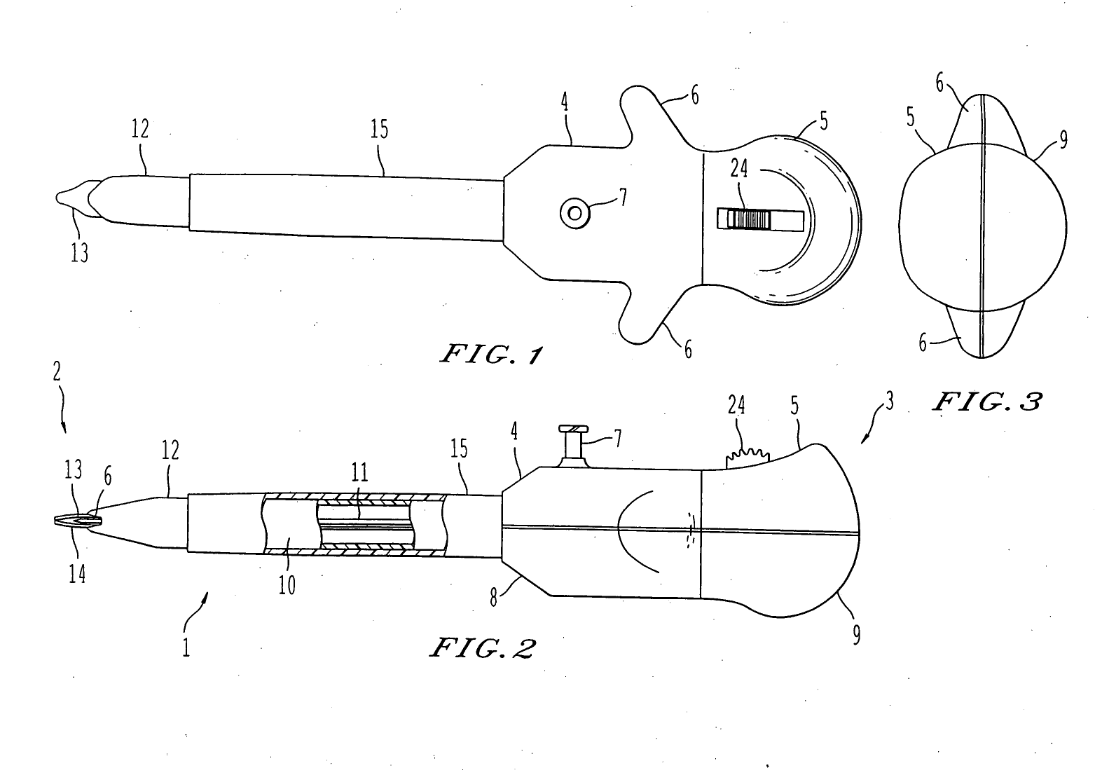

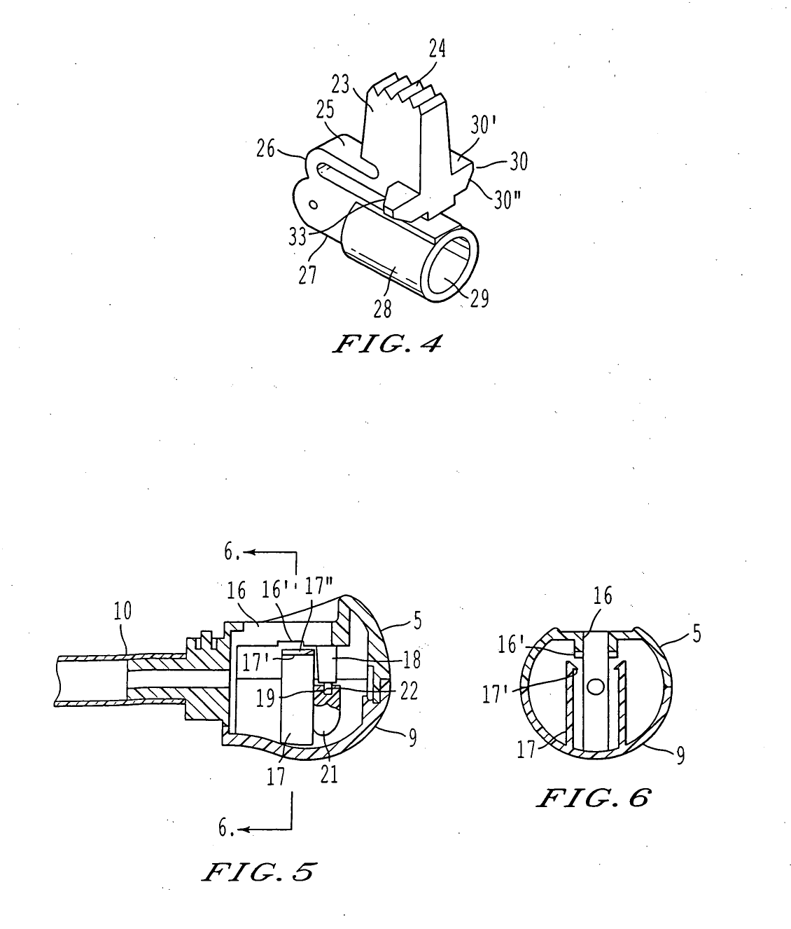

Referring to the drawings enclosed here, FIG. 1 is a top plan view, FIG. 2 is a side view, and FIG. 3 is a proximal end view of the outside of a trocar 1 according to a preferred embodiment of the present invention. FIGS. 1-3 offer a context for the details of the locking system inside the handle. FIGS. 1-3 show a trocar 1 having a distal end 2 and a proximal end 3 and is provided with a blade 6 housed within safety guard tips 13, 14. While the preferred embodiment utilizes a metal blade, it is understood that any sharpened, substantially flat member made of another material such as plastic or a composite material could be utilized. The main element of the mechanism is shown in FIG. 4 which is called the integrated actuator button and latch module 23. FIG. 5 shows the inside of the proximal end of the handle without some of the internal mechanisms to facilitate initial understanding of the context space. FIG. 6 shows a cross-section taken along line 6-6 of FIG. 5 looking toward the left as indicated by the arrows. The flexible hold-downs 17, 17 are shown attached to the housing at their bottom end.

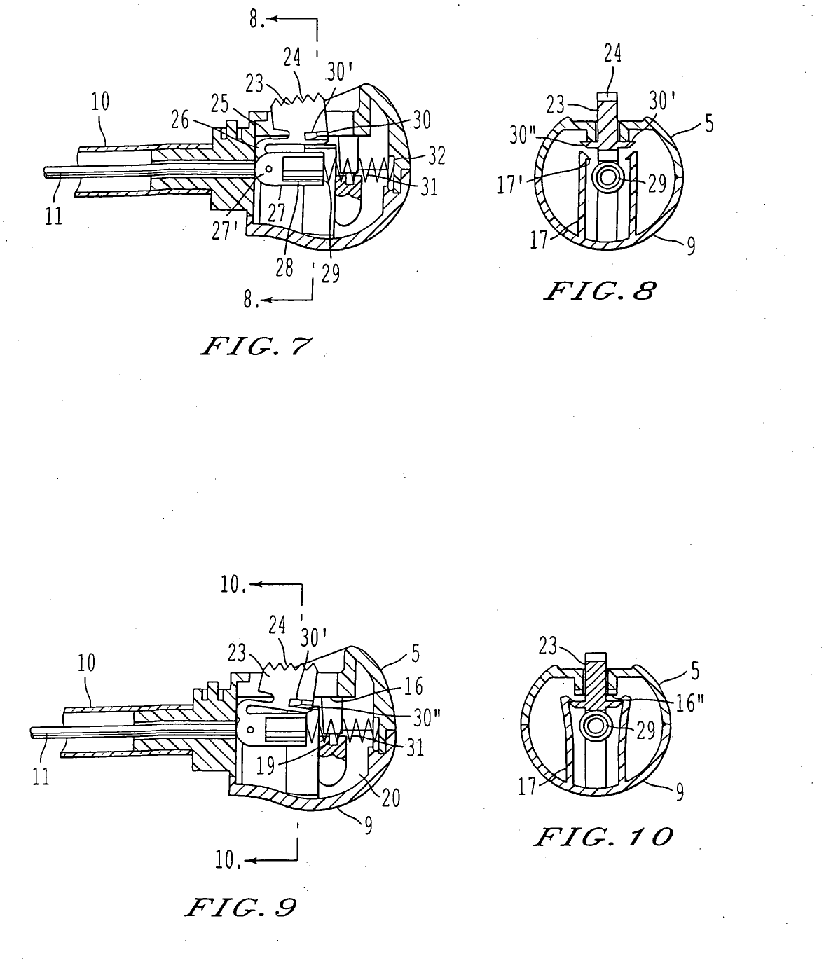

FIG. 7 shows the complete mechanism within the inside of the handle described above. The system is shown here in its initial, or rest, position prior to actuation. FIG. 8 shows the inside of the mechanism as seen in the direction of section arrows 8-8 in FIG. 7. FIG. 9 shows the internal configuration of the mechanism right after the actuation set up, or arming. The actuator button 23 as shown has been pushed down and it is now free to move. The actuator latch is now out of engagement with a locking notch 16′ and is free to move to the right as shown in FIG. 9. FIG. 10 shows the cross-section taken along line 10-10 in FIG. 9. FIG. 11 shows an enlarged detail of the ridge of a hold-down 17, 17 while holding the latch in the free-to-move position detached from its locking notch 16.

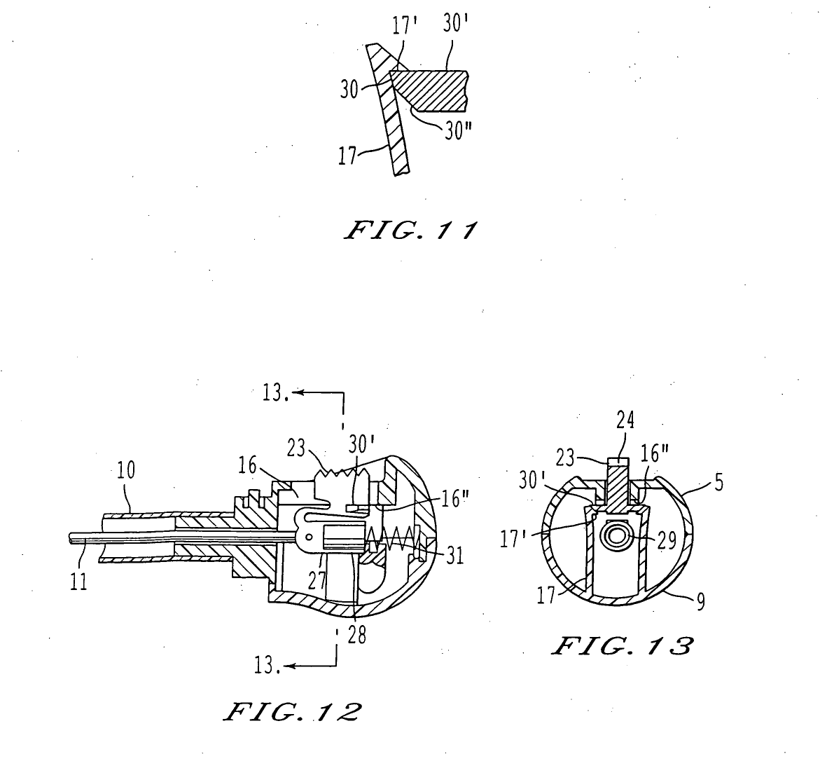

FIG. 12 shows the entire actuator button and locking system 23 sliding toward the right after snapping off and shifting upwards from the hold-downs 17, 17 and against the bottom sides of its guide groove 16″. In that position the actuator button and latch module 23 is able to return under the action of a spring 31 toward the left and lock back into its locking notch 16, thereby relocking the whole mechanism at its initial position, as shown in FIG. 7.

As shown in FIGS. 1, 2, and 3, element 4 is the top of the cannula handle containing the luer 7 and attached to cannula 15. Element 12 comprises the tip expander of a penetrator tube 10. Safety guard tips 13 and 14 are provided as shown. Inside of the cannula 15 are the penetrator tube 10 and the actuator or obturator shaft 11. Another important element is the actuator button 23 fitted into the groove on the top portion 5 of the proximal housing/handle. The bottom parts 8, 9 of the handle are on the distal end side and on the handle proximal end.

In FIG. 4 is shown the important elements that integrate the actuator button 23 and the latches 30. The button 23 is connected through spring portions 25 and 26 to the hub 27 that is attached to the actuator shaft 11, and to the spring housing 28 and spring guide bore 29.

FIG. 5 shows the horns 6 of the trocar and the elements of the penetrator handle housing portion 5 and part 9 which together form the housing for the whole actuator module 23. The housing portion 5 of the proximal handle also forms a support for the penetrator tube 10. The housing bottom engages the top portion 5 forming a substantially spherical proximal end well fitted for being grasped by a surgeon's hand. The proper engagement between the top portion 5 and the bottom 9 of the handle housing is insured by a pin 22 which protrudes from a downwardly extending stud 18 which protrudes from the top portion 5 and fits into a hole 19 in a second stud 21 at the interim portion 20 of the bottom member 9. That insures precise alignment between the two halves of the handle end. The bottom member 9 also holds the two hold-downs 17, 17 protruding into the top space inside 5, and having protruding rims 17′, 17′ that in turn engage the surfaces 30′ of the latches 30, 30 at the sides of the actuator button 23 shown in FIG. 4 (not shown here). The hold-downs having protruding rims 17′, 17 at their tops also have those rims 17′, 17′ which are beveled with bevels 17″, 17″, respectively, toward the inside to avoid interference with the passing back and forth of the moving button latches when they are not being held down.

At the top of handle housing 5 on FIG. 4 there is a guide space formed between walls 16, 16 to guide the actuator button 23. The guide between the walls 16, 16 has a lower edge thereof notched as shown by notch 16′ to serve as a locking notch for the actuator latches 30, 30 which spring into them to lock the system and prevent motion of the actuator system. That is the purpose for the locking notch 16′ being provided.

FIG. 6 shows the inside of the handle in section as seen from the right in the direction of the arrows in line 6-6 appearing in FIG. 5. The walls 16 at the sides of the guide have the notch 16′ as shown in FIG. 6 to provide the spaces for the locking latches 30, 30 of the actuator button to lock into them at the start or end of each work cycle. As seen in FIG. 6 the tops of the hold-downs 17′ are tilted some 45° toward the inside to match the slope 30′ of the latches 30, 30 that will engage them and spread them apart when the button 23 is depressed, and the locking is effected when the hold downs rims 17′, 17′ snap over them. Such action will be discussed in greater detail hereinbelow.

FIG. 7 shows the full actuator mechanism assembled and in the locked position. The actuator shaft 11 is shown inserted into the hub 27 and a small shear pin 27′ is inserted across the hub to fix it in place. A coil spring 31 is inserted into the bore 29 at the right end of the spring housing cylinder 28. The squared end of the spring 31 is inserted into a seat 32 at the junction between the two handle halves 5 and 9. The left end of the actuator including spring portion 26 will contact the frontal inside wall of the top portion 5 of the housing and prevent any further leftward movement. The spring portion 25 of the actuator button module will be slightly depressed forcing the actuator button 23 upward and the latches 30, 30 into their locking notches 16 such that no axial motion will be possible.

FIG. 8 shows the inside of the system as seen from the right side across the section plane 8-8 of FIG. 7. In this figure the button 23 is shown fully protruding above its guide plane. The serrated edge 24 is highly visible. The top of the latch arms 30′, 30′ are fully inserted into the locking notches 16′ and the system is locked onto the guide at each side. At this time there is no contact between the actuator button and locking module shown in FIG. 4 and the two hold-down arms 17, 17′. This is the normal locked position of the system prior to arming it.

FIG. 9 shows what happens when the actuator button 23 is pushed down into the housing. The spring portion 25 of the module bends downward and the locking latch surfaces 30′, 30′ are forced down against the top of the hold-down top ring 17′ tilted 45° inwardly, and the module 45° angle latches 30″, 30″ slide against them forcing them to open as shown in FIGS. 10 and 11 until the tips of the latches 30 pass down and the ridges 17′, 17′ snap and click inwardly over surface 30′ catching it as shown in FIG. 11 and holding it away from the locking notch 16′. In that position the system is said to be armed since it can be axially moved by any force applied inwardly against the tip of the safety guards, as when starting a penetration.

FIG. 12 shows what happens when an inward force is applied to the tip of the safety guards. Since in the armed position there is no structure to stop the motion (there is no locking latch inserted into a notch 16′ because the latches are being held down away from the lower edge of the guides 16), the whole actuator button 23, along with the entire sensing system of safety guards and actuator shaft 11 moves to the right until the latches lose contact with the hold-down ridges 17′, 17′ and snap out and against the lower edge of the guides 16″, 16″ but far to the right of the locking notch 16′, so the module is free to either move right, or return to the left.

In the position shown in FIG. 13, the module latches 30′, 30′ slide freely against the lower edge of the guides 16″, 16″ while still touching the slanted tops of arms 17, 17 with a slight contact against them, but easily sliding. The hold-down arms 17, 17 shown in FIG. 13 do not close down completely but keep slightly in touch with the latches 30, 30 until the latches return and reach the locking notch 16′ and snap to lock again at the end of each cycle. To minimize the extent of the initial deflection of the spring 25 which might have to deflect too much from a large gap between latches and hold-downs at the start, the frontal bevels 33 shown facilitate the sliding between the latches 30, 30 and the top of the hold-down ridges 17′, 17′ while upon return to the locking direction. It has been found that the system described here represents the best mode of operation since at no time is there any slack between parts which might induce a malfunction. All parts are in sliding contact throughout critical functions.

FIG. 5 shows another optimization of arms 17, 17 wherein is shown a slight bevel 17″ at the right edge of ridge 17′. The presence of bevel 17″ will facilitate easy entrance of the bevel 33 at the distal edge of the latches 30 on the return trip. Those two latches 30 could also have conical surfaces at the front and bottom instead of the pyramidal structure shown. The choices depend on the materials chosen and their coefficients of friction, and that is more a question of manufacturing preference than inventive requirement. It is also entirely possible to design this system for avoiding contact between the returning latches and the hold-downs, and by so doing also avoid need for the frontal ring 17″ and bevel 33. However, in the interest of good engineering practice the configuration described represents the best mode and that is why it is preferred.

Basically, the actuation system described here represents an important set of ideals. In the first place, it is the simplest approach to the design of locking systems for disposable medical devices which could be easily reset if so desired. This system is characterized by an integration of functions that are usually far more complicated. The most important element of this system is the actuator button with locking latches, integral spring, locator for driving shaft, housing for external spring, and functional indicator with visual, tactile and acoustic clues. Altogether eight functions in one single element. The rest of this system require simple modifications of parts that already exist in all similar instrument housings, which means that with the insertion of a single new part all the rest of the required functions are obtained. This may sound like an exorbitant claim but it is a physical fact easily verifiable and is hardly contestable.

Obviously, numerous modifications and variations of the present invention are possible in light of the above teachings. It is therefore to be understood that within the scope of the appended claims, the invention may be practiced otherwise than as specifically described herein.

1. A surgical device, comprising:
a cutting blade;
a guard portion movable with respect to the blade from a first position covering the blade to a second position exposing the blade;
an actuator shaft extending along an axis of the surgical device from a first end connected to the guard portion to a second end;
a biasing element;
an integrally formed locking element; and
a handle having a cavity configured to receive the locking element.

2. The surgical apparatus recited in claim 1, wherein the integrally formed locking element includes an actuating portion, a biasing portion, a hub, a guide portion, and a bore, the hub is connected to the second end of the actuator shaft, the biasing portion is configured to bias the actuating portion in a direction away from the longitudinal axis of the surgical device, and the bore is configured to receive the biasing element to bias the locking element towards the first position where the guard portion covers the blade.

3. The surgical apparatus recited in claim 2, wherein the actuating portion is movable toward the axis of the surgical device by an operator, the handle includes a hold-down element and a locking notch, the locking notch being configured to receive the guide portion of the locking element when the actuating portion is not pressed toward the axis and hold the guide portion in the first position, the hold-down element being configured to hold the guide portion of the locking element when the actuating portion is pressed toward the axis, and wherein the locking element is configured to slide from the first position to the second position when the guide portion of the locking element is held by the hold-down element.

4. The surgical apparatus recited in claim 3, wherein the hold-down element comprises a hold-down member located on at least one side of the axis.

5. The surgical apparatus recited in claim 4, wherein each hold-down member includes a protruding rim.

6. The surgical apparatus recited in claim 5, wherein each protruding rim includes a first beveled surface that contacts a bottom surface of the guide portion when the actuating portion is pressed toward the axis.

7. The surgical apparatus recited in claim 6, wherein the first beveled surface forms an angle of substantially 45° with a top surface of the guide portion.

8. The surgical apparatus recited in claim 6, wherein the bottom surface of the guide portion is beveled.

9. The surgical apparatus recited in claim 8, wherein the beveled surface of bottom surface of the guide portion forms an angle of substantially 45° with a top surface of the guide portion.

10. The surgical apparatus recited in claim 9, wherein each protruding rim includes a second beveled surface that contacts a front surface of the guide portion when the locking member moves from the second position to the first position.

11. The surgical apparatus recited in claim 10, wherein the second beveled surface forms an angle of substantially 45° with a back surface portion of the guide portion.

12. The surgical apparatus recited in claim 11, wherein the front surface of the guide portion is beveled.

13. The surgical apparatus recited in claim 12, wherein the front surface of the guide portion forms an angle of substantially 45° with a back surface of the guide portion.

14. The surgical apparatus recited in claim 5, wherein each protruding rim includes a beveled surface that contacts a front surface of the guide portion when the locking member moves from the second position to the first position.

15. The surgical apparatus recited in claim 14, wherein the beveled surface forms an angle of substantially 45° with a back surface of the guide portion.

16. The surgical apparatus recited in claim 14, wherein the front surface of the guide portion is beveled.

17. The surgical apparatus recited in claim 16, wherein the front surface of the guide portion forms an angle of substantially 45° with a back surface of the guide portion.

18. The surgical apparatus recited in claim 3, wherein the biasing portion biases the actuating portion away from the axis such that the guide portion is biased towards the locking notch.

19. The surgical apparatus recited in claim 3, wherein the hold-down element extends along the axis such that the guide portion is released by the hold-down element before the locking element reaches the second position.

20. The surgical apparatus recited in claim 1, wherein the handle includes a first portion including a pin and a second portion including a hole for receiving said pin when the first portion is fixed to the second portion.

21. The surgical apparatus recited in claim 2, wherein the actuating portion includes a serrated edge.

22. A method of using a surgical device having a penetrator, a locking element, a locking notch and an actuation portion, and a guard portion movable with respect to said penetrator, comprising:
depressing said actuating portion to remove a guide portion of said locking element from said locking notch;
moving said locking element from a first position to a second position to uncover a penetrator protected by said guard portion;
piercing a membrane of a patient with the penetrator; and
moving said locking element from the second position to the first position to cover the penetrator with the guard portion.

23. The method of using a surgical device recited in claim 22, wherein depressing the actuator portion comprises depressing a biasing portion of the locking element which integrally connects the actuating portion and the locking element.

24. The method of using a surgical device recited in claim 23, wherein depressing of the actuator portion comprises depressing the actuating portion until the guide portion is received by a hold-down element in a handle of the device.

25. The method of using a surgical device recited in claim 24, wherein moving the locking element from a first position to a second position comprises sliding the guide portion of the locking element along a longitudinal axis of the surgical device while the guide portion is held by the hold-down element.

26. The method of using a surgical device recited in claim 25, wherein moving the locking element from a second position to a first position includes releasing the guide portion of the locking element from the hold-down element.

27. The method of using a surgical device recited in claim 25, wherein piercing the membrane includes releasing the guide portion of the locking element from the hold-down element.

28. A method of assembling a surgical device having an obturator shaft and a handle having a first and a second handle portion, comprising:
attaching an integrally formed locking element to an obturator shaft;
inserting a biasing element into a hub of the locking element;
placing the locking element in said first handle portion with one end of the biasing element facing a surface of said first handle portion; and
connecting said second handle portion to said first handle portion over the locking element.

29. The method of using a surgical device recited in claim 28, wherein connecting a second handle portion to the first handle portion comprises inserting a pin in said first handle portion into a hole in said second handle portion.

30. The method of using a surgical device recited in claim 28, wherein connecting a second handle portion to the first handle portion comprises inserting a pin in said second handle portion into a hole in the first handle portion.
